(19) United States
(12) Patent Application Publication
(10) Pub. No.: US 2020/0360604 A1
(43) Pub. Date: Nov. 19, 2020

(54) Gravity Infusion Control System

(71) Applicant: Shift Labs, Inc., Seattle, WA (US)

(72) Inventors: Beth Elise Kolko, Seattle, WA (US); Koji Intlekofer, Seattle, WA (US); Michael Connolly, Ferndale, WA (US); David Stephen Zucker, Seattle, WA (US)

(73) Assignee: Shift Labs, Inc., Seattle, WA (US)

(21) Appl. No.: 16/876,420

(22) Filed: May 18, 2020

Related U.S. Application Data
(60) Provisional application No. 62/849,287, filed on May 17, 2019.

(51) Int. Cl. A61M 5/168; A61M 39/28; A61M 39/22; G01F 1/66; G01G 3/14; G01G 19/18

(52) U.S. Cl. CPC A61M 5/1689; A61M 39/28; A61M 39/22; A61M 5/16895; A61M 2205/3584; G01G 3/14; G01G 19/18; A61M 2205/52; A61M 2205/502; G01F 1/661

(57) ABSTRACT

A monitoring device provided for monitoring the delivery of fluids through a drip chamber. The device includes an electromagnetic radiation source and a radiation detector. A weight detector is positioned to determine the weight of a fluid reservoir over time, and the detector is positioned with respect to the drip chamber to detect drops falling through the drip chamber. A processor is coupled to the radiation detector and the weight detector to determine flow rate, and is further coupled to a flow restrictor to selectively increase or decrease flow based on the determined flow rate.

### PRIORITY CLAIM

This application claims the benefit of U.S. provisional application No. 62/849,287 filed May 17, 2019, the contents of each of which are incorporated by reference.

### FIELD OF THE INVENTION

The present disclosure relates generally to the delivery of intravenous fluids, more specifically to the monitoring of the delivery of fluids intravenously to a subject.

### BACKGROUND OF THE INVENTION

When delivering fluids intravenously, healthcare professionals typically focus on the volume delivered per unit time. For example, a physician might prescribe that a drug be diluted in saline solution and the mixture administered at a specific number of milliliters per hour. While IV pumps that can dose IV fluid with high precision are effective, they are out of the financial reach of many facilities, and either operate with AC power or have short battery lives. Gravity infusion is an inexpensive method to administer IV fluids, and at its cheapest does not require any sort of power. However, it is typically not possible to obtain an accurate flow rate due to human error, manufacturing variation, fluid viscosity, and other factors.

Gravity infusion typically involves hanging a reservoir of IV fluid from a pole, and connecting a disposable tubing set (also called a dripset) to that reservoir, which is typically a bag or bottle, via a spike on the tubing set and puncture membrane port on the reservoir. Somewhere along the tubing, normally quite close to the spike, there is an aperture from which the fluid can fall, and a space with diameter much larger than the tubing, where fluid can accumulate these comprise the drip chamber. Tubing is affixed to the bottom of the drip chamber, and typically, the tubing sets also include some sort of restriction device (e.g. a roller clamp) to limit the flow through the tube.

The time scale of import for fluid delivery is typically on the order of minutes. That is to say, as long as fluid is flowing at approximately the correct rate, it does not matter if the patient receives a little too much medication one minute and too little the next, as long as the average flow rate is correct.

Tubing sets are categorized by manufacturers with a gtt rating, which provides an estimate of the volume of each drop produced. For example, a 60 gtt tubing set has an aperture in the drip chamber that is supposed to form drops that are each 1/60th of a milliliter. Put another way, 60 drops from a 60 gtt tubing set should make exactly 1 milliliter. To achieve a target flow rate in mL/hour, the end user would multiply the rate by the gtt rating and divide by 240 to get the number of drops per every 15 seconds. They would then adjust the flow restriction device on the tubing, and count the number of drops in 15 seconds, and repeat the adjustment until the desired number of drops per unit time is falling. Alternately, an electronic drop counter can be used to more accurately count drops.

Because the adjustment process is quite finicky and time consuming, it would be desirable to have a device automatically perform flow restriction adjustments. Unfortunately, the gtt ratings are approximate, and the true drop size varies based on a number of parameters, including: manufacturing variations in the plastic parts, flow rate, and fluid viscosity (which in turn varies due to drug properties). Manufacturing variation alone can account for 10% variation in drop size. This error results in inaccurate fluid delivery, even when using an electronic drop counter with automatic flow control.

### SUMMARY OF THE INVENTION

The present disclosure provides devices, methods, and systems for providing real time monitoring of a fluid flow rate and an accumulated total volume through a drip chamber, and control of the fluid flow rate.

In one version, the monitoring device for monitoring fluid passing from a fluid reservoir and through a drip tube of a tubing set, includes a detector positioned to receive electromagnetic radiation from an emitter and to generate a detector signal in response to the electromagnetic radiation received from the emitter. The emitter and detector are positionable about the tubing set, whereby the electromagnetic radiation passes through the drip tube from the emitter to the detector

Preferably, a processor is in communication with the detector, with a memory having stored programming instructions operable by the processor to detect a plurality of fluid drops passing through the drip tube based on the detector signal.

In one version, a mechanical flow restrictor is positioned in-line with the tubing set.

Preferably, a weight sensor is coupled to the fluid reservoir and in communication with the processor, the weight sensor being configured to detect a weight of the fluid reservoir when the fluid reservoir is hanging in suspension;

The memory preferably includes stored programming instructions to cause the mechanical flow restrictor selectively increase or decrease fluid flow through the tubing set as a function of a change in detected weight of the fluid reservoir.

In some versions, the stored programming instructions are further operable by the processor to filter sources of noise acting against the weight sensor.

In some versions, the stored programming instructions are further operable by the processor to determine a flow rate as a function of the detected plurality of fluid drops.

In some versions, the stored programming instructions are further operable by the processor to determine a flow rate as a function of the change in detected weight.

In some versions, the stored programming instructions are further operable by the processor to determine a flow rate as a function of the detected plurality of fluid drops and the change in detected weight.

In a preferred version, the mechanical flow restrictor is a clamp.

In other preferred versions, the mechanical control restrictor is a valve.

Preferably, the weight sensor detects a plurality of weights of the fluid reservoir at a plurality of times, and the processor causes the memory to store the plurality of weights and the plurality of times.

In some versions, the weight sensor is a strain gauge.

In some versions, the weight sensor is integrally mounted to the fluid reservoir.

Preferably, the monitoring devices includes a display and the memory comprises stored programming instructions causing the display to present the determined flow rate.

### BRIEF DESCRIPTION OF THE DRAWINGS

Preferred and alternative examples of the present invention are described in detail below with reference to the following drawings.

FIG. 1 illustrates an exploded view of a flow rate monitoring device with a drip chamber and collar for attachment of the drip chamber to the device.

FIG. 2 illustrates a front elevational view of a representative fluid reservoir or bag attached to a tubing set, with a portion of a tube further extending from the tubing set.

FIG. 3 is a representative illustration of the bag and tubing set of FIG. 2, further shown in position hanging from a hook and with a scale or strain gauge attached.

FIG. 4 illustrates a preferred monitor having an integrated flow rate controller.

FIG. 5 illustrates a preferred version of a flow rate controller.

FIG. 6 illustrates an alternate preferred version of a flow rate controller.

FIG. 7 illustrates an alternate preferred version of a flow rate controller.

FIG. 8 is a block diagram of a gravity infusion control system, including a monitor, scale, and fluid reservoir with tubing.

### DETAILED DESCRIPTION OF THE PREFERRED EMBODIMENT

A preferred monitoring system consists of a monitoring unit which interfaces mechanically or electrically with a drip set. Preferably, a sensor is arranged to detect drops falling within a drip chamber in order to monitor fluid flow. In this system, the monitoring unit may receive sensor values and perform continuous or other monitoring of the infusion. More preferably, a weight sensor or strain gauge is used with a fluid reservoir or IV bag to monitor changes in weight of the fluid reservoir, and therefore changes in fluid within the bag, as a further aid in monitoring fluid flow.

One preferred version of a monitoring unit or controller may be in the form of an all-in-one device configured for monitoring fluid flow, including by selectively constricting or enabling fluid flow to thereby adjust the fluid flow rate. In one version, a retractable tool head having a terminal abutment may be actuated to apply pressure to the tubing set, effectively pinching the tubing and limiting the cross sectional area available for fluid flow when the tool head is pressed against the tubing. In other versions the internal area devoted to fluid flow is reduced in a different fashion other than by applying pressure or “pinching.” Preferably, the actuator is moveable in either direction to selectively restrict or increase the fluid flow through the tubing. Thus, the movement of the tool head toward and against the tubing may be reversed to withdraw the tool head and reduce or remove the pinching effect, thereby opening the internal area within the tubing in the vicinity of the tool head and allowing greater flow.

A user interface allows for a desired flow rate to be indicated. A user can input into the controller a variety of settings including, but not limited to, the length of time an infusion should take place, the volumetric flow rate the fluid should flow, or the total volume of fluid to be allowed to flow through the controller. A microprocessor operates on stored programming instructions responsive to user input to indicate a desired flow rate, and then determines if the flow rate must be increased or decreased based on the current measured flow rate, causing the actuator to be adjusted accordingly, either by moving in a direction that pinches the tubing set more to reduce flow, or moving in the opposite direction to increase flow. By repeating this process continuously, repeatedly comparing the user-established input and controlling the actuator as necessary, the flow rate could be moderated.

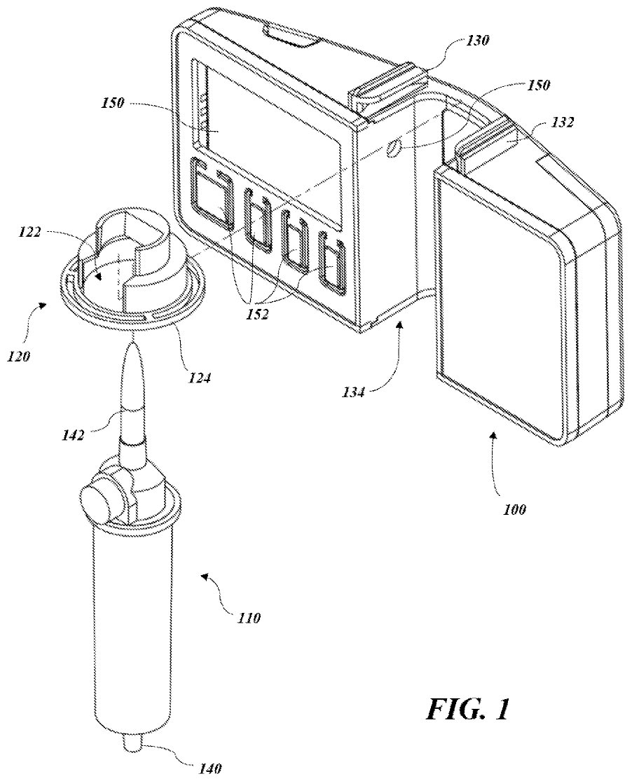

One example of a gravity flow controller is shown in FIG. 1. As illustrated, the controller or monitoring device 100 mounts onto the drip chamber 110 of an infusion tubing set, with a tubing set including a collar 120 that fits into mounting rails 130, 132 on the controller to ensure proper alignment and secure the controller onto the drip chamber. The tubing 140 extends beyond (and beneath, as illustrated) the drip chamber 110.

In some versions, the monitoring device 100 may include an emitter or source 150 such as an infrared emitting diode, or IRED. An opposing detector may be positioned in opposition along the inner surface of a trench 134 as described in U.S. Pat. No. 10,456,522, the entirety of which is incorporated by reference. When the tubing set is mounted in the trench, the source and detector are positioned about the tubing set 110 in such a way so that drops falling from the top to the bottom of the tubing set will at least partially obscure or refract the beam which extends along a line of sight from the source or emitter to the detector.

The monitoring device 800 may include display unit 150, user input interface 152, and user audio interface such as a speaker.

The tubing set is illustrated with an exemplary upper receiving tube 142 having an upper end in the form of a spike for insertion into a fluid source such as fluid source as discussed further below. A fluid reservoir or bag attaches to the spike, but is not illustrated in FIG. 1 for simplicity of illustration.

In one version, the collar 120 includes an upper opening 122 to allow the spike 142 and an upper portion of the tubing set to pass through the opening as the collar surrounds the upper portion of the tubing set. The collar also includes a peripheral radially-extending flange 124. Preferably, the flange 124 is in the form of an annular flange which surrounds the collar and encircles the tubing set when attached to the tubing set. The collar may be permanently attached to the tubing set, or in other versions may be removably attachable.

In operation, the tubing set is mounted to the monitoring device, preferably with the IRED and PD mounted on the monitoring device so that a line of sight is provided through the drip tube, fixedly mounted in a proper location, diametrically opposite one another. As described above, the tubing set may further be secured in place either with a mechanical latch, magnets, other mechanical features.

In the version illustrated in FIG. 1, the channels 130, 132 of the monitoring device 100 are positioned at the top of the device, formed in an opposing pair of brackets which extend toward one another, and in a direction toward the trench in each case. The flange 124 is configured to be snugly received within the channels in order to firmly hold the collar 120 and tubing set 110 in place within the trench 134. Most preferably, the interior shape of the channels within the brackets is in the form of a mating arc corresponding to the shape of the annular flange, such that the annular flange will be snugly and frictionally held within the channels, and further in which the annular flange can rotate while being held within the channels while holding the tubing set and drip tube in the same position with respect to its central axis.

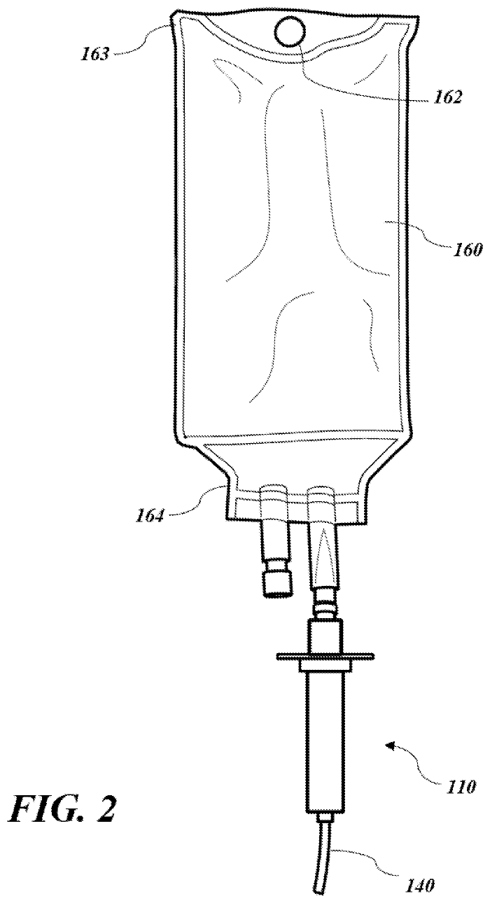

FIG. 2 illustrates a representative fluid reservoir or IV bag 160, in this case shown attached to the tubing set. A hole 162 is provided at the top 163 of the bag for attachment to a hook so that the bag may hang and allow gravity to urge the fluid to the bottom 164 of the bag. The controller or monitoring device illustrated in FIG. 1 is not shown in FIG. 2, for clarity of illustration, but it should be appreciated that the tubing set 110 may be mounted to a controller in the same manner as illustrated in FIG. 1.

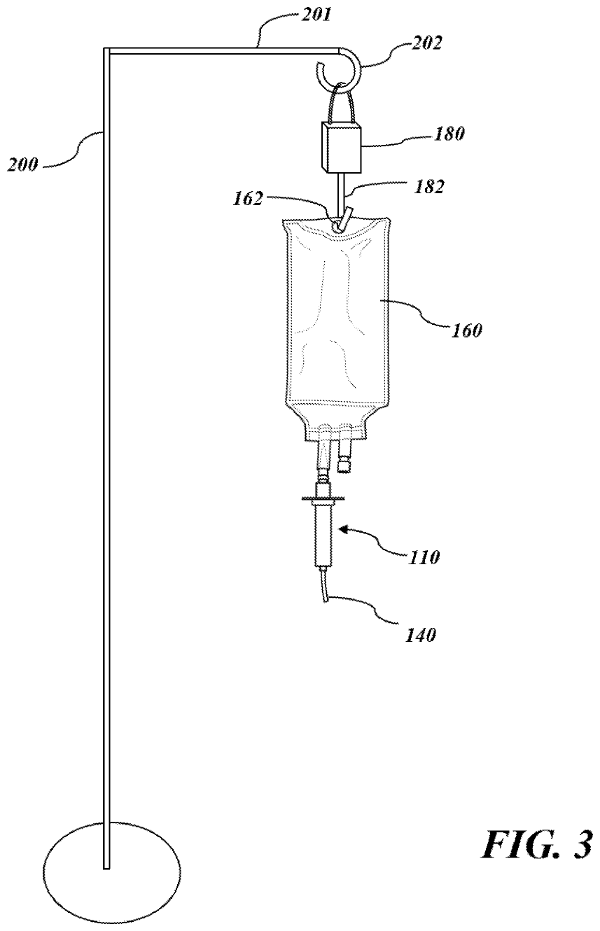

FIG. 3 illustrates a fluid reservoir and tubing set (again, shown without a controller, for clarity) hanging from a hook 182 of a weight sensor or scale 180. The scale in turn is suspended from a hook 202 of a stand 200. The stand is merely representative, and may be configured differently. Likewise, the scale 180 need not be suspended from a hook, but rather could be integrally formed with a bag support structure, attached to a stand or structure that is not a “hook,” or otherwise incorporated into a system for use with a fluid reservoir of the type described here. For example, it may be integrated into or adhered to the horizontal arm 201 of the stand 200, rather than being suspended from a hook as a separate component. In yet other versions, a strain gauge or other form of weight sensor may be incorporated into the bag itself, preferably in the vicinity of the hole, in order to detect changes in weight over time.

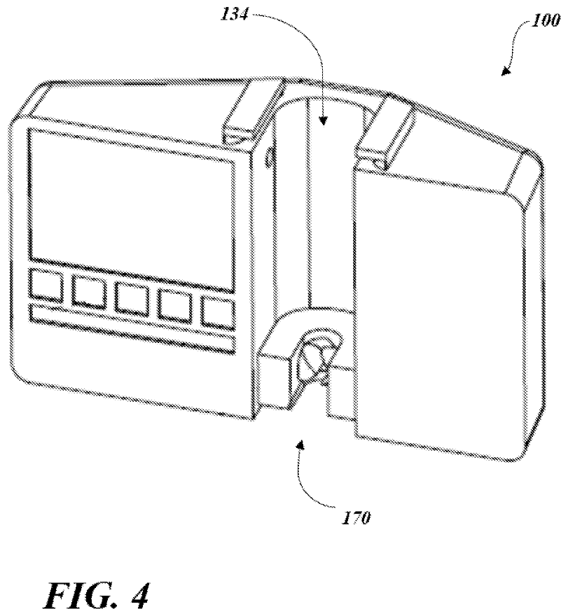

FIG. 4 illustrates an alternate version of a controller 100. As with the controller described above, the tubing set is received within the trench 134. The lower portion of the tubing 140 (see FIGS. 1-3) is received within a gap between a mechanical flow constrictor such as a clamp 170. In the illustrated example, the clamp is located at a lower portion of the controller, beneath the tubing set when the tubing set is installed within the trench. In yet other versions, the clamp may be located at a higher or different location on the controller.

In general, the mechanical clamp includes a head driven by a stepper motor, servo, or solenoid toward an anvil or other surface within the trench or gap formed in the lower portion of the controller. The tube is inserted into the clamp so that a lower portion of the tube abuts the clamp and is sandwiched between the head and the anvil. The head is configured to be actuated to move toward and against the tube (preferably orthogonally with respect to an axis extending through the tube) to perform a pinching (moving into the tubing) or releasing (moving away from the tubing) motion to increase or decrease the amount of fluid that can flow through the tubing set.

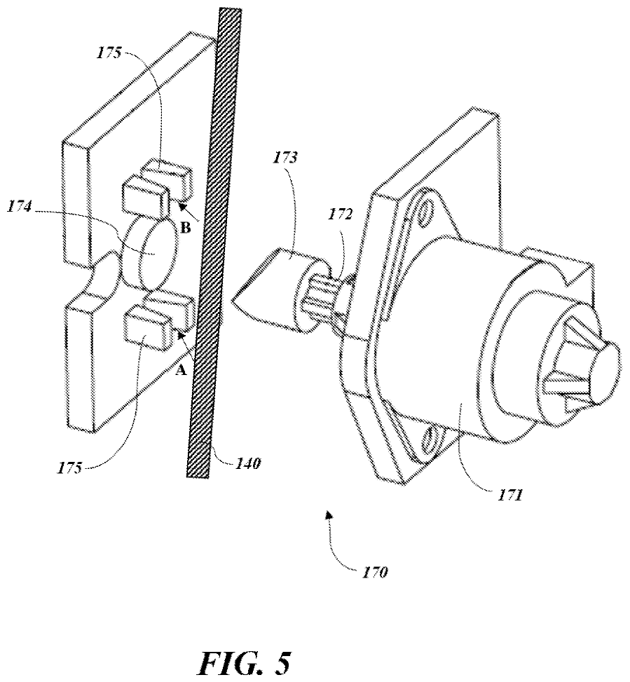

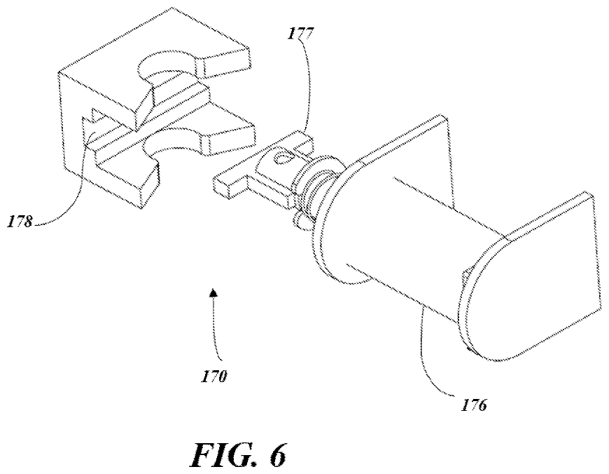

FIGS. 5 and 6 illustrate representative preferred versions of restrictors or clamps suitable for use with the controller as described above. With reference to FIG. 5, a stepper motor 171 is connected to a lead screw 172 which provides linear motion to drive the lead screw toward or away from the tube 140. A head 173, which is preferably formed as a wedge-shaped tip, is positioned at the end of the screw and provides a focused region of force into the lower tube. As the lead screw is extended, the lower tube is pinched between the extending tip and an anvil or solid surface 174, reducing the cross-sectional area and reducing flow rate. The area defined between the head and the anvil defines the gap as referred to above. Optionally, a first side of the clamp may include clips 175 to retain the tube 140 in position against the anvil 174. The tube may be attached to the clips by pressing the tube into the clips in the direction of arrows A and B in FIG. 5.

In certain embodiments, a force sensor may also be incorporated to provide additional data to the controller. Because gravity infusion is variable from moment to moment due to factors external to the gravity infusion system, data should be generated and processed in an ongoing basis in order for the controller to maintain an accurate rate. In one such embodiment, the sensor would be located on the anvil 174 or other surface against which the tubing is being pressed. The force sensor could be positioned over the anvil, or behind it, provided the anvil is allowed to move laterally with the direction of the force being applied by the tip 173.

An alternate clamp 170 is illustrated in FIG. 6. In this example, a solenoid 176 is used to allow or prevent the flow of fluid through a tubing set by pinching and releasing the tubing set. The end of the solenoid's armature contains a head 177 sized and arranged to pinch the lower tube when pressed against it. In this example, the head is more flattened than the version of FIG. 5. The servo would extend or retract as needed to allow short intervals of fluid flow, or dictate when individual fluid drops fall through the drip chamber. This rate at which the solenoid opened to allow drops or fluid to flow could then be used to set the desired flow rate communicated by the microprocessor. An anvil or solid surface 178 supports the tubing set and prevents the lower tube from moving during actuation.

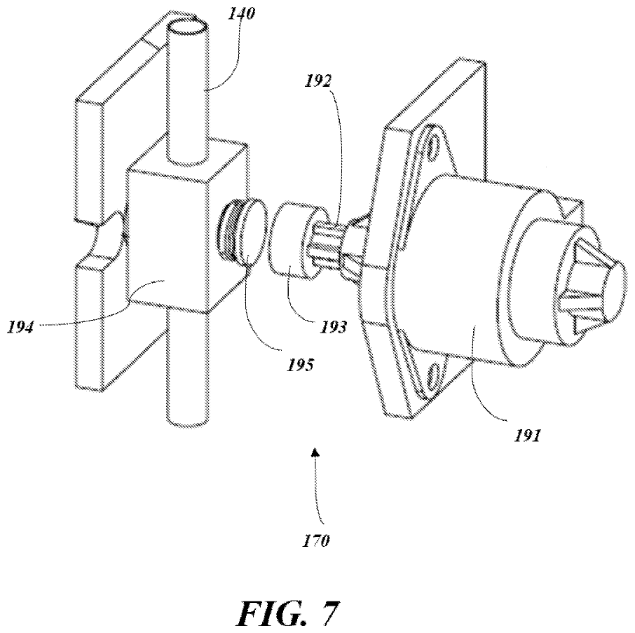

In another embodiment of the system, as illustrated in FIG. 7, the actuator may push against a mechanical valve integrated into the tube, rather than pressing against the tube itself. In this case, a motor 191 connected to an actuator arm or lead screw 192 pushes a plunger 193 positioned within the controller. A tubing set containing a mechanical component in-line with the fluid and tube is then inserted into the controller opposite the plunger. The mechanical component 194 contains a spring-loaded stem of a valve 195 that the plunger 193 pushes against. This valve system is in-line with the fluid flow and tube 140. The valve components are preferably configured as part of the tubing set, separate from the controller. To limit flow rate, the plunger pushes towards the stem, closing the valve. The valve may be a globe or needle valve design, or yet other forms of valves operable to control flow through a tube. Inconsistent and changing flow rates are seen in plastic tubing due to the stretching, relaxing, and flexing of the plastic material as it is deformed. Providing this rigid mechanism eliminates the issues of plasticity of the tubing set. The actuator can move the valve stem in either direction to increase and decrease flow rate through the tube based on what the user specified as the desired flow characteristics. If the controller measures the flow to be different than the user input value and this information sent to the microprocessor. The software on the microprocessor then determines if the flow rate needs to be increased or decreased, and sends a signal to the actuator to move the plunger forward or backward to achieve the desired flow change. An acceptable error between the measured flow rate and the user-input flow rate would be defined. To reduce the need for frequent and power-draining adjustments, the flow rate can alternate between a high and low state relative to the desired rate to achieve the desired flow rate as an average over a specified amount of time.

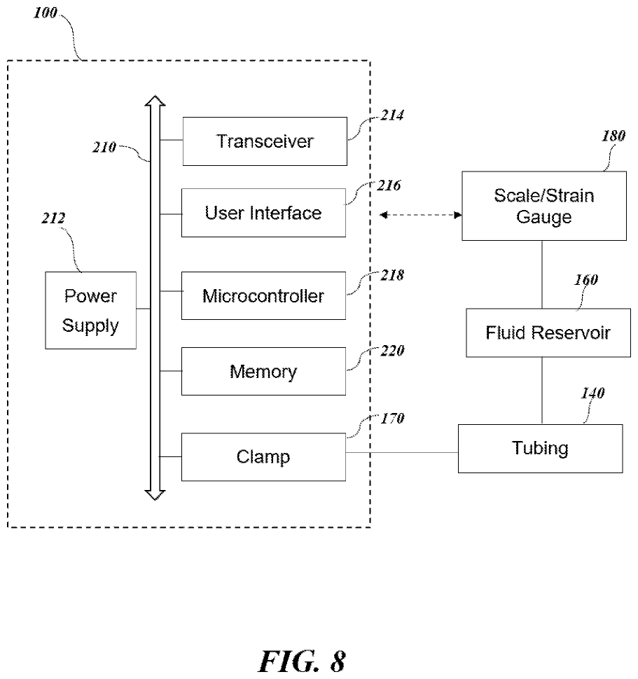

FIG. 8 illustrates a block diagram of a preferred implementation of a flow rate monitoring system. The controller 100 includes a power supply 212, which may be in the form of batteries or AC, and which powers the other components of the controller. Though a single power supply is illustrated, more than one power supply may be provided for the different components. A transceiver 214 is included with the controller, and configured to wirelessly communicate with the scale or strain gauge 180 and optionally with a remote monitoring unit. In other versions, the communication in each case may occur in a wired configuration. The strain gauge is mounted to the fluid reservoir 160, which leads to the tubing 140 and is preferably connected to a clamp 170 of any of the types described above. The controller further includes an internal processor device such as microcontroller 218 with associated memory 220. A user interface 216 may include the screen, buttons, and speaker, as described above. A communications bus 210 allows for communicative coupling between components as shown.

As used herein, the term “processor device” is not intended to be limiting. Rather, as used throughout the present disclosure, processor device may refer to one or more devices enabled to execute instructions that perform actions. In some embodiments, a processor device may receive input and provide corresponding output in response to the received input. In some embodiments, processor device may include a programmable microcontroller. In some embodiments, a processor device may include a microprocessor. A processor device may include a field programmable gate array (FPGA). In other embodiments, a processor device may include an application specific integrated circuit (ASIC). In some embodiments, a processor device may include a computer and/or a mobile device. In some embodiments, a processor device may include a processing core, memory, input/output peripherals, logic gates, Analog-to-Digital Converters (ADCs) and such. In some embodiments, a processor device may include a plurality of processor devices in communication with one another across a network or a bus.

The microcontroller, or processor device, optionally with a separate memory, contains stored programming instructions operable by the microcontroller to monitor fluid flow rate as a function of drip count. In a fashion as described above, and preferably in accordance with the description of U.S. Pat. No. 10,456,522, the controller monitors flow rate by detecting drops falling through the tubing set, using the photo emitter and detector. Thus, the flow monitoring may consist of an infrared light source and a photodiode mounted on opposing sides of the drip chamber so that the beam crosses through the path of a falling drop. The microprocessor may periodically measure the light sensed by the photodiode and, by sensing the change in detected light, can detect that a drop has fallen. By measuring the elapsed time between detected drops, the microprocessor can measure the drop rate and calculate flow rate.

In addition to the measurement modality explained above, multiple other types of data may be generated by the system. Each type of data may play a different role in determining how the software on the microprocessor does its calculations depending on the desired infusion rate and materials used. Variables may include slower versus faster flow rates and level of accuracy of tubing sets. The software on the microprocessor incorporates each of these data points at different time intervals and uses them to calculate what instructions should be sent to the actuator that allow the actuator to maintain required flow rate. Some data may be collected at milliseconds, some may be collected and only assessed over time intervals. There may be additional data that is collected at second or more intervals. Based on the integration of these multiple data types, the software on the microprocessor sends instructions to the actuator to achieve the required flow rate.

For example, at low flow rates with less accurate tubing sets, data from a strain gauge may be the primary information used to make calculations for the instructions that are sent to the actuator. At higher flow rates, drop detection may play a primary role. Different types of data may be weighted differently in importance by the software as it processes information. The algorithms used to make calculations and send instructions to the actuator will be dynamic and responsive to elements outside the system such as flow rate mentioned above. Thus, the feedback generated by the multiple measurement and sensing modalities in the controller will be fed into the algorithms and processed according to needs as determined by usage scenario.

Certain embodiments include a strain gauge as a peripheral attachment to the device that would be hung between the fluid bag and the mounting point, as described above. The gauge measures the axial force exerted by the bag on the hanging point. The purpose of the strain gauge is to provide additional information on the momentary fluctuations in flow rate. Fluctuations can be caused by a variety of reasons such as changing position of the tubing line or lowering fluid level and pressure. These, and other changes, may affect the flow rate, but not the fluid mass in the bag. Periodic measurements of the force exerted on the poll by the bag provide information on the change in fluid volume. This information provides ongoing data points that are used by the microprocessor of the controller to process data in a continuous algorithm, communicating to the actuator mechanism to adjust its position to affect the flow rate in the desired way. By measuring total bag weight at each moment in time, the strain gauge provides a more accurate reading of fluid volume than the approach of integrating rate information over time. Measuring bag weight does not accrue additional error over time, providing a useful secondary-check on the total volume measurements for the device. It generates data that is processed both as readings at the moment in time, as well as readings over time intervals. The strain gauge can be connected to the main device via a wire or wireless link.

As flow rate data is collected or calculated such as described above, the microprocessor compares the determined flow rate with the user-input rate, and determines if a higher or lower flow rate is needed to achieve the user-input value. Adjusting flow rate (in a downward or reducing direction) can be achieved by constricting the flow allowed through the tubing of an infusion set, including by an instruction from the processor to open or close the clamp as may be appropriate. The opening or closing of the clamp would then be expected to alter the flow rate, which in turn would be detected by the drip rate emitter and detector, and further confirmed by the strain gauge readings. Over time, this continual adjustment of the flow rate by the clamp, based on input from the strain gauge and the drip detector, ensures a more consistent flow rate.

Preferably, the microcontroller is in signal communication with the strain gauge or other sensor, and tracks the change in fluid bag status over time. This data preferably is stored in a memory accessible by the microcontroller. The microcontroller also stores the setting of the linear actuator and the corresponding drip rate intended by the user input settings. At predetermined intervals (which may be user-adjusted, in some versions), the microcontroller compares the flow (either in terms of flow rate or total weight or volume) as determined by the fluid bag sensor with the flow as input by the user-settings. If the two disagree by an undesirable amount (which may be set by a user, or pre-programmed in memory), then the microcontroller will cause the actuator to increase or decrease the flow accordingly, so that the desired flow is achieved. In one version, the volume as measured in the fluid bag is taken to be accurate, and when the fluid bag sensor determines that the actual flow exceeds the user-input flow rate, the linear actuator is adjusted accordingly to reduce or increase the flow into alignment.

In some examples, the strain gauge (or other sensor) data may be stored in the associated memory over time, and used to adjust the relationship between the linear actuator position and the user-input flow rates, in order to improve the accuracy of the user-input over time.

In certain embodiments, a force sensor may also be incorporated to provide additional data to the controller. Because gravity infusion is variable from moment to moment due to factors external to the gravity infusion system, data must be generated and processed in an ongoing basis in order for the controller to maintain an accurate rate. In one such embodiment, the sensor would be located on the surface the tubing set is being pushed into by the actuator.

As the data is fed to the microprocessor, algorithms process the data in such a manner as to overlay moment-in-time readings with readings over time intervals. Measuring the force that the tip of the actuator pushes against the tubing provides information on how that force is changing at any given instant; this information is processed in conjunction with the time-interval readings. The actuator may move in to reduce flow. The force actuator might also move back in order to increase flow. For example, because of the behavior of the plastics in the gravity infusion system, the plastic tubing will relax against the actuator after an undetermined interval. This will cause the force that is exerted to change, and this change in force will be registered by the force sensor. This will then be communicated to the software on the microprocessor. The software on the microprocessor will process this information, potentially in conjunction with additional data sources from the system, and then communicate instructions to tell the actuator to adjust its position inward or outward. The instructions sent by the microprocessor will be based on data from multiple sensing modalities, including potentially drop detection and flow detection.

This force sensor can also be used as a fail-safe indicator in the event the actuator moves too far in one direction. In the event feedback from the actuator to the force sensor indicates the force has increased to a level exceeding safety or otherwise desired forces that feedback will be read by the microprocessor and instructions will be communicated to the actuator to reduce clamping force.

In accordance with a further preferred aspect of the invention, the controller is configured to filter out sources of error, such as sensor noise. The weighing measurement of the scale or strain gauge as described above introduces new sources of error, such as sensor noise, quantification error in an analog-to-digital converter, mechanical oscillations in the system, and human-introduced motion (i.e. the patient moves around, which in turn moves the tubing attached below the drip chamber, moving the reservoir itself).

To account for error in the flow rate measurement due to drop volume variation (and other noise) and error in the mass measurement, a filter algorithm provides an estimate of flow rate that is more accurate than either measurement on its own could provide. The preferred algorithm is a Kalman filter, which accomplishes the task without overburdening computing requirements (extending battery life). A Kalman filter is a recursive process that estimates the state of a linear dynamic system based on a model of the system and periodic measurements of that system. It takes as inputs a set of predetermined coefficients (relating to the variance of the state variables as well as the variance of the measurements) as well as the periodically updated control set point and the periodic measurements.

The Kalman Filter includes two mathematical models of the system: one pertaining to changes in the state of the system (the motion model); and the other pertaining to changes in the measurements (the measurement model).

In a preferred implementation, the state of the system is described using a three-dimensional vector:

$$x = ⌈ \begin{matrix}v \\ δ \\ {\dot{v}}_{d}\end{matrix} ⌉$$

where ν is the volume of liquid remaining the reservoir (measured in L), δ is the error in flow rate (measured in L/s) defined as δ=\dot{ν}_{d}−ν, and \dot{ν}_{d} is the desired flow rate into the patient (a constant over the infusion, measured in L/s). We deliberately choose to include the error in flow rate, rather than flow rate directly, since our control step requires a term that goes to zero when our goal is reached. In this case, δ will be zero when our estimated mean flow rate matches our target flow rate.

As the Kalman filter is a discrete-time algorithm, we choose a time period Δt that is sufficiently long to allow process changes to occur (in our case, more than a couple seconds), but sufficiently short so corrections can be made to the set point before any adverse effects (in our case, less than a minute). A value of 5-10 seconds is appropriate for a gravity infusion with standard dripset tubing.

The motion model is governed by the linear equation:

xₜ=Δxₜ₋₁+Buₜ+ϵₜ,ϵₜ˜N(0,V)

### Where:

xₜ represents the state at current time step t

A is a constant 3×3 matrix

$$⌈ \begin{matrix}1 & - Δ t & - Δ t \\ 0 & 1 & 0 \\ 0 & 0 & 1\end{matrix} ⌉$$

xₜ₋₁ represents the state at previous time step t−1

B is a constant 3×1 matrix

$$⌈ \begin{matrix}0 \\ Δ t \\ 0\end{matrix} ⌉$$

uₜ is the rate of change in control setpoint (in units L/S²)

And ϵₜ represents the process noise in the system; a normal distribution of mean 0 with variance V, a 2×2 matrix determined empirically as described below.

The variance matrix V reflects uncertainty about the time evolution of the system, and is equal to

$$[ \begin{matrix}{σ}_{vol}^{2} & 0 & 0 \\ 0 & {σ}_{drop}^{2} & 0 \\ 0 & 0 & 0\end{matrix} ] .$$

To find σᵥₒₗ, measure the difference between expected and actual volume given a set of initial conditions, and calculate the standard deviation. To find σ_{drop}, measure the difference between expected and actual flow rates and calculate the standard deviation. Given the difficulty of precisely controlling flow rate (for example due to the elasticity of the tubing or plastic creep in the valve), variance of drop count is relatively high, whereas the volume should be fairly predictable.

And the measurement model is governed by the equation:

zₜ=Cxₜ+ωₜ,ωₜ˜N(0,W)

### Where:

zₜ is the 2×1 measurement state vector

$$[ \begin{matrix}{M}_{t} \\ {d}_{t}\end{matrix} ] ,$$

where Mₜ is the measured mass (in grams) less the static estimated mass of the plastic bag, tubing set, drop counting device, and tubing; and dₜ is the number of drops counted during the most recent time period Δt

C is the constant 2×3 observation matrix

$$[ \begin{matrix}ρ & 0 & 0 \\ 0 & \frac{Δ t}{P} & \frac{Δ t}{P}\end{matrix} , ]$$

where ρ is the fluid density measured in g/L and P is the approximate volume of a drop (as determined by the gtt rating, measured in L).

And ωₜ represents the measurement noise, a normal distribution of mean 0 with variance W, a 2×2 matrix described below.

The variance matrix W is the constant matrix

$$[ \begin{matrix}{σ}_{scale}^{2} & 0 \\ 0 & {σ}_{count}^{2}\end{matrix} ] .$$

σ_{scale} is the standard deviation of the measurement of the mass, and can be determined by recording a series of mass measurements from the system with a fixed weight and calculating the standard deviation. σ_{count} is the standard deviation of the drop counting measurement, and can be similarly determined empirically.

The automatic flow restriction mechanism, for example, a wedge pinching the tube against a flat anvil, is driven by a motor with its own control system. It is assumed that there is a transfer function F between a Kalman filter set point measured in L/s and a motor control set point (measured in steps of a stepper motor or encoder counts, to provide two examples). This transfer function need not be linear, and can be determined in advance. Similarly, the control algorithm used to guide the motor to the set point could be as simple as sending step pulses to a stepper motor, or as complicated as a PID loop on a servomotor with an encoder.

The power of the Kalman filter comes from two key properties of Gaussian distributions: that linear combinations of Gaussian variables are also Gaussian; and that given an observation of one part of a multivariate Gaussian variable and the covariance of the components, one can compute the mean and variance of the rest of that Gaussian variable.

With A, B, C, W, and Δt determined, each iteration of the algorithm happens every Δt seconds, and involves several steps:

1. Commanding the flow restriction mechanism to set point F(Sₜ) which is calculated by Sₜ=Sₜ₋₁+uₜΔt, where uₜ is determined by the control algorithm (described later).

2. Calculating the motion update:

μ⁻=Aμ⁺+Bu

Σ⁻=AΣ⁺A^{T}+V

Where:
μ⁻ represents the prior mean
μ⁺ is the previous posterior mean, or, in the special case of the first iteration, the matrix described below
Σ⁻ represents the prior variance
Σ⁻ is the previous posterior variance, or, in the special case of the first iteration, the matrix described below
And A^{T} is the transpose of the matrix A.

3. Measuring the mass (M), subtracting the static estimated weight; and recording the number of drops (d) since the last measurement

4. Calculating the measurement update:

μ⁺=μ⁻+L(z−Cμ⁻)

Σ⁺=(I−LC)Σ⁻

Where:
μ⁺ is the new posterior mean; this value is the new estimate of the state of the system, a two-dimensional vector composed of the estimated actual volume of liquid remaining the reservoir, and the estimated actual flow rate of liquid through the tube during the current time period
L is a 3×2 matrix equal to Σ⁻C^{T}(CΣ⁻C^{T}+R)⁻¹; C^{T} is the transpose of the matrix C
z is the 2×1 measurement state vector

$$[ \begin{matrix}M \\ d\end{matrix} ] ,$$

containing the most recent mass measurement M and drop count d
Σ⁺ is the new posterior variance matrix, which roughly equates to the uncertainty in the posterior means

For the first iteration of a given infusion, the values of ρ⁺ and Σ⁺ represent the initial system state conditions and initial variances, respectively, and are set as follows:

$${μ}^{+} = [ \begin{matrix}{v}_{init} \\ 0\end{matrix} ] , {\sum }^{+} = [ \begin{matrix}{σ}_{init}^{2} & 0 & 0 \\ 0 & 0 & 0 \\ 0 & 0 & 0\end{matrix} ]$$

Where the initial volume νᵢₙᵢₜ is calculated by dividing the average of several mass measurements, less the average mass of a dripset plus tubing plus drop counting device, by the density of the fluid. And σᵢₙᵢₜ² is the variance in initial volume of liquid, determined empirically by measuring the mass of a sample of fluid reservoirs, converting to volume using the density, and calculating the standard deviation. As we expect no fluid to be flowing initially (since the flow restrictor is fully closed), the variance in the flow rate is zero, as is the variance of the constant target flow rate.

The outputs from the Kalman filter (i.e. the periodically updated posterior means and posterior variances) are used to update the control set point of the automatic flow restrictor. This control can be calculated by a variety of methods, but the preferred algorithm is a discrete-time linear-quadratic-Gaussian (LQG) controller. As we will show, the LQG controller in this case is actually equivalent to a proportional controller (the “P” term of PID control) on the value of the state.

The LQG controller seeks to minimize:

$$J = {\sum }_{t = 0}^{N} ( {x}_{t}^{T} {Qx}_{t} + {u}_{t}^{T} {Ru}_{t} )$$

### Where:

Q is a 3×3 matrix containing all zeros except the center element, which is the relative “cost” of changing the flow
R is a scalar which is the relative “cost” of adjusting the control.

As we will see, these two numbers ultimately serve only to adjust the feedback constant for our controller. Their scales are arbitrary (hence they matter only relative to one another), and some trial and error will be required to find values that result in a well-controlled system.

Once the desired flow rate has been set, we use A, B, Q, and R to calculate the infinite time horizon feedback gain matrix K_{∞}.

K_{∞}=(B^{T}S_{∞}B+R)⁻¹B^{T}S_{∞}A

Where S_{∞} is the solution to the discrete algebraic Riccati equation:

S_{∞}=A^{T}(S_{∞}−S_{∞}B(B^{T}S_{∞}B+R)⁻¹B^{T}S_{∞})A+Q

This is not solvable with our chosen state (since we include a constant in the state matrix, it is not controllable) and with our chosen Q (which is not a positive definite matrix). This equation is solved numerically instead, using fixed point iteration. The above equation is calculated iteratively until the difference between each previously-calculated and newly-calculated matrix value falls below a predetermined threshold. An example threshold would be 1e−10.

Once we have calculated S_{∞}, we can then calculate K_{∞}. A sample K_{∞} matrix is [0 0.18849 0], where the middle element of the matrix is the feedback coefficient that scales the estimated δ to a new set point u.

To incorporate the controller into our Kalman filter loop, we insert a step prior to step 1, applying K to μ⁺ to get new set point:

i=−Kμ⁺

In operation, the system is preferably used as follows. First, a user hangs a fluid reservoir bag on strain gauge-enabled hook, connects the dripset, primes the line, and enables a mechanical flow restrictor (or clamp) on the tubing. The user positions a control monitor of the type described above, with drop counting and control, around the drip chamber and tubing. Automatic flow control starts out completely closed.

The user initially disables the mechanical flow restrictor or clamp, and connects the tubing to a cannula in a patient. The user then interacts with the device via an interface (e.g. buttons and LCD) and sets some infusion parameters applicable to the particular fluid and desired flow rate. In one example, the user enters the gtt rating of the tubing, and sets the desired infusion flow rate.

Optionally, for a more accurate infusion, the user may input fluid density (since it does change from drug to drug) or select from a set of commonly used drugs (in this case, the controller memory will contain stored densities of those drugs).

Optionally, the user indicates they wish to receive certain alerts, such as if/when the flow rate drops below a certain threshold (e.g. 50% of target rate), or when the remaining fluid volume is below a certain threshold (e.g. 30% of original volume).

Optionally, the user can indicate what conditions will end the infusion (e.g. a total volume delivered, the flow rate set point value falling above or below a certain threshold, or the flow rate dropping to zero).

The user then indicates they wish to begin infusion. Preferably at this stage, the processor computes the initial μ⁺ and Σ⁺ matrices as described above. As further described in the Kalman Filter and Control sections above, the processor software repeatedly performs the filter and control steps every Δt seconds to: (a) Apply the control equation to calculate new set point u; (b) Translate the new set point z from flow rate to actuator space using transfer function F, and use the motor control software/hardware to command flow restrictor to its new set point; (c) Perform the Kalman filter motion update, computing μ⁻ and Σ⁻; (d) Sample strain gauge to obtain new mass and store the number of drops detected since last measurement; (e) Perform the Kalman filter measurement update, computing new μ⁺ and Σ⁺; (f) Optionally, update the display of some or all of the process variables or other derived values (such as estimated flow rate, drops per Δt, total volume of fluid delivered so far, etc.); (g) Optionally, alert the user if any of the alert conditions are met; and (h) Optionally, fully close the automatic flow restrictor when the infusion end conditions are met and alert the user.

The user can also terminate the infusion at any point by using the interface or simply applying the manual flow restrictor to the tubing.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

We claim:

1. A monitoring device for monitoring fluid passing from a fluid reservoir and through a drip tube of a tubing set, comprising:
an emitter mounted on the monitoring device;
a detector mounted on the monitoring device;
the emitter being positioned to emit electromagnetic radiation toward the detector;
the detector being positioned relative to the emitter to receive to receive the electromagnetic radiation from the emitter and to generate a detector signal in response to electromagnetic radiation received from the emitter;
the emitter and detector being positionable about the tubing set, whereby the electromagnetic radiation passes through the drip tube from the emitter to the detector;
a processor in communication with the detector;
a memory having stored programming instructions operable by the processor to detect a plurality of fluid drops passing through the drip tube based on the detector signal, and further to determine a flow rate for the fluid based on the detected plurality of drops;
a mechanical flow restrictor in-line with the tubing set; and
a weight sensor coupled to the fluid reservoir and in communication with the processor, the weight sensor being configured to detect a weight of the fluid reservoir when the fluid reservoir is hanging in suspension;
the memory further having stored programming instructions to cause the mechanical flow restrictor selectively increase or decrease fluid flow through the tubing set as a function of a change in detected weight of the fluid reservoir and the detected plurality of drops over a period of time.

2. The monitoring device of claim 1, wherein the stored programming instructions are further operable by the processor to filter sources of noise acting against the weight sensor.

3. The monitoring device of claim 2, wherein the mechanical flow restrictor is a clamp.

4. The monitoring device of claim 2, wherein the mechanical control restrictor is a valve.

5. The monitoring device of claim 4, wherein the weight sensor detects a plurality of weights of the fluid reservoir at a plurality of times, and the processor causes the memory to store the plurality of weights and the plurality of times.

6. The monitoring device of claim 2, wherein the weight sensor is a strain gauge.

7. The monitoring device of claim 2, wherein the weight sensor is integrally mounted to the fluid reservoir.

8. The monitoring device of claim 1, further comprising a display and wherein the memory comprises stored programming instructions causing the display to present the determined flow rate.

9. A monitoring device for monitoring fluid passing from a fluid reservoir and through a drip tube of a tubing set, comprising:
a detector positioned to receive to receive electromagnetic radiation from an emitter and to generate a detector signal in response to the electromagnetic radiation received from the emitter;
the emitter and detector being positionable about the tubing set, whereby the electromagnetic radiation passes through the drip tube from the emitter to the detector;
a processor in communication with the detector;
a memory having stored programming instructions operable by the processor to detect a plurality of fluid drops passing through the drip tube based on the detector signal;
a mechanical flow restrictor in-line with the tubing set; and
a weight sensor coupled to the fluid reservoir and in communication with the processor, the weight sensor being configured to detect a weight of the fluid reservoir when the fluid reservoir is hanging in suspension;
the memory further having stored programming instructions to cause the mechanical flow restrictor selectively increase or decrease fluid flow through the tubing set as a function of a change in detected weight of the fluid reservoir.

10. The monitoring device of claim 9, wherein the stored programming instructions are further operable by the processor to filter sources of noise acting against the weight sensor.

11. The monitoring device of claim 9, wherein the stored programming instructions are further operable by the processor to determine a flow rate as a function of the detected plurality of fluid drops.

12. The monitoring device of claim 9, wherein the stored programming instructions are further operable by the processor to determine a flow rate as a function of the change in detected weight.

13. The monitoring device of claim 9, wherein the stored programming instructions are further operable by the processor to determine a flow rate as a function of the detected plurality of fluid drops and the change in detected weight.

14. The monitoring device of claim 10, wherein the mechanical flow restrictor is a clamp.

15. The monitoring device of claim 10, wherein the mechanical control restrictor is a valve.

16. The monitoring device of claim 10, wherein the weight sensor detects a plurality of weights of the fluid reservoir at a plurality of times, and the processor causes the memory to store the plurality of weights and the plurality of times.

17. The monitoring device of claim 10, wherein the weight sensor is a strain gauge.

18. The monitoring device of claim 17, wherein the weight sensor is integrally mounted to the fluid reservoir.

19. The monitoring device of claim 10, further comprising a display and wherein the memory comprises stored programming instructions causing the display to present the determined flow rate.
